(19) United States
(12) Patent Application Publication
(10) Pub. No.: US 2004/0005281 A1
(43) Pub. Date: Jan. 8, 2004

(54) Artificial nail attachment method and a solution used therefor

(75) Inventor: Takehiko Ohara, Osaka-shi (JP)

Correspondence Address: KODA & ANDROLIA, 2029 CENTURY PARK EAST, SUITE 1430, LOS ANGELES, CA, 90067-3024, US

(73) Assignee: Ohara Shoji Co., Ltd.

(21) Appl. No.: 10/612,736

(22) Filed: Jul. 2, 2003

(30) Foreign Application Priority Data
Jul. 2, 2002 (JP) 2002-193389

(51) Int. Cl. A61K 7/04

(52) U.S. Cl. 424/61

(57) ABSTRACT

A method for attaching an artificial nail to a fingernail including the steps of coating the fingernail with an aqueous type thermoplastic resin coating material, and then bonding an artificial nail to the fingernail by an instantaneous adhesive agent from above the thermoplastic resin coating material after the thermoplastic resin coating material has dried into a film. The thermoplastic resin coating material includes 1 to 5 wt % of polyvinyl alcohol added to an ethylene-vinyl acetate copolymer emulsion.

### BACKGROUND OF THE INVENTION

1. Field of the Invention

The present invention relates to a method for attaching an artificial nail to one's own natural fingernail and a solution used for such a method.

2. Prior Art

Conventionally, an artificial nail is attached to a natural fingernail using a two-sided adhesive agent, a tacky adhesive agent or a strong instantaneous adhesive agent.

The former two methods that use two-sided adhesive agents and tacky adhesive agents have several problems. Since the bonding strength is weak, the artificial nail is easily stripped off in cases where the artificial nail catches on some object or in cases where the artificial nail is exposed to even light work (such as washing) in water. In order to avoid these problems, an instantaneous adhesive agent that has a strong bonding strength is used. However, with such an adhesive agent, due to the strong bonding strength, there are difficulties in the removal of the artificial nail.

More specifically, since fingernails constantly grow, leaving the artificial nail on the natural fingernail interferes with the growth of the fingernail. In addition, the tip of the fingernail tends to become dirty. Accordingly, it is necessary to remove the artificial nail after a certain period of time has elapsed.

Conventionally, when removing an artificial-nail that has been attached by an instantaneous adhesive agent, a remover such as a solvent, acetone or MEK (Methyl Ethyl Keton), has been employed. However, since the bonding strength is strong, time is required in order to strip away the artificial nail; and since the pulling force is inevitably applied when the artificial nail is removed, pain is felt in the natural fingernail. Thus, the problem is that removal of the artificial nail entails pain, and the artificial nail, which is usually expensive, is damaged.

Furthermore, since an instantaneous adhesive agent is applied directly to the natural fingernail, even if the artificial nail is removed, the fingernail tends to be damaged, and roughness of the fingers is caused by the remover.

### SUMMARY OF THE INVENTION

Accordingly, the object of the present invention is to provide an artificial nail attachment method that makes it possible to fasten the artificial nail firmly to a natural fingernail (merely called “fingernail”) and also makes it possible to remove such an artificial nail without scratching the fingernail or damaging the artificial nail.

The above object is accomplished by a unique process of the present invention that includes the steps of coating the fingernail with an aqueous type thermoplastic resin coating material, and then, after the thermoplastic resin coating material has dried, bonding an artificial nail to the fingernail with an instantaneous adhesive agent from above the thermoplastic resin coating material.

The above object is further accomplished by a unique solution of the present invention that is used for attaching an artificial nail to a fingernail, in which the solution is a thermoplastic resin coating material and is comprised of 1 to 5 wt % of polyvinyl alcohol added to an ethylene-vinyl acetate copolymer emulsion. It is only necessary to coat the surface of the fingernail with this coating material as in the form of a manicure. When the coating material dries, this coating material becomes clear, thus making it possible to confirm that the coating material has dried.

The film, which is formed on the surface of a fingernail by the thermoplastic resin coating material, is superior in terms of adhesion to the fingernail. Furthermore, when an artificial nail is bonded to the surface of this coating material by an instantaneous adhesive agent, the artificial nail is firmly fastened to the fingernail, so that ordinary daily activities or light work in water will not cause any stripping of the artificial nail. Moreover, a direct bonding between the fingernail and the instantaneous adhesive agent is prevented by the above-described film of the thermoplastic resin coating material.

The film of the thermoplastic resin coating material is a thermoplastic and aqueous type film. Accordingly, the coating film will become soft and can dissolve if the finger tip is put in hot water at 42° C. to 43° C. for approximately five minutes. Accordingly, the artificial nail can be removed without scratching the fingernail; and since there is no damage occurs to the artificial nail, the artificial nail can be used repeatedly.

### BRIEF DESCRIPTION OF THE DRAWINGS

FIG. 1A shows, in a longitudinal cross section, the fingernail coated with a thermoplastic resin coating material, and FIG. 1B is a partially cut-away top view thereof; and

FIG. 2A shows, in a longitudinal cross section, the fingernail with the artificial nail attached thereon using the film of a thermoplastic resin coating material and an instantaneous adhesive agent, and FIG. 2B is a partially cut-away top view thereof.

### DETAILED DESCRIPTION OF THE INVENTION

Embodiments of the present invention will be described below with reference to the accompanying drawings.

In the artificial nail attachment method of the present invention that is to attach an artificial nail 2 to a fingernail 1, the fingernail 1 is first coated with an aqueous type thermoplastic resin coating material 3; then, after this thermoplastic resin coating material 3 has dried, the artificial nail 2 is bonded to the fingernail 1 by an instantaneous adhesive agent 4 from above the film of the thermoplastic resin coating material 3.

There are no particular restrictions on the material of the thermoplastic resin coating material 3 as long as this material has the characteristics of forming a film that is strongly fastened to the surface of the fingernail 1 when this coating material 3 is applied to the fingernail 1 and of softening and dissolving when this coating material 3 is immersed in warm water for a certain period of time. The coating material 3 can be formed by way of mixing 1 to 5 wt % polyvinyl alcohol with an ethylene-vinyl acetate copolymer emulsion. When such a thermoplastic resin coating material 3 is applied to the surface of a fingernail 1 as in a manner of manicure application and dried, this coating material 3 becomes clear, thus making it possible to confirm that the coating material 3 has dried. Furthermore, the coating material 3 has sufficient water solubility so that the coating material 3 will soften and dissolve when immersed in hot water of 42° C. to 43° C. for approximately five minutes. Instead of ethylene-vinyl acetate copolymer emulsion, modified styrene-butadiene copolymer latex and/or denatured acetic acid polyvinyl copolymer resin can be mixed so as to improve the strength (adhesive force) and water-resisting properties and heat resisting properties.

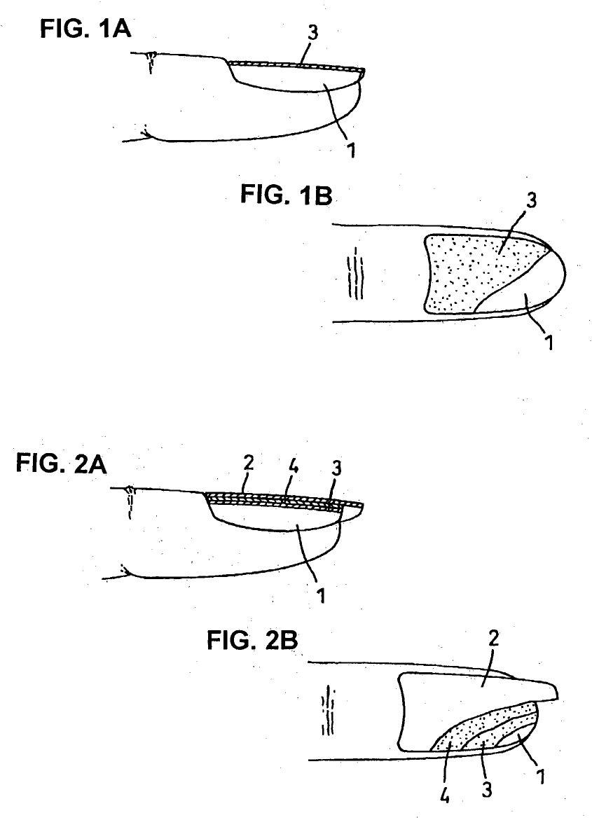

In order to attach the artificial nail 2 to the fingernail 1, the aqueous type thermoplastic resin coating material 3 is applied to a uniform thickness over the entire upper surface of the fingernail 1 by the same procedure as in a manicure application as shown in FIGS. 1A and 1B. When this thermoplastic resin coating material 3 dries, the coating material 3 becomes clear film; and thus it is possible to confirm that the coating material 3 has dried.

Afterward, the artificial nail 2 is bonded to the surface of the film of the coating material 3 using an instantaneous adhesive agent 4 as shown in FIGS. 2A and 2B, and the artificial nail 2 is fastened to the fingernail 1 or to the film of the thermoplastic resin coating material 3. The film of the thermoplastic resin coating material 3 is superior in terms of adhesion to the fingernail 1; and by way of bonding the artificial nail 2 to the surface of such a film by an instantaneous adhesive agent 4 that has a strong bonding strength, the artificial nail 2 is strongly fastened to the fingernail 1. As a result, an attachment strength that is sufficient to prevent stripping of the artificial nail by daily activities or light work in water can be obtained.

When it is desired to remove the artificial nail 2, the fingertip with the artificial nail 2 attached is immersed in hot water. In other words, immersion of the fingertip for approximately five (5) minutes in hot water at 42° C. to 43° C. can cause the film of the thermoplastic resin coating material 3 to be softened and dissolved; as a result, the artificial nail 2 is easily removed without scratching the fingernail 1. Since the artificial nail 2 is undamaged, it can be reused. Moreover, since no remover or removing solution is used, there is no resulting roughness of the fingers.

In the above description, the artificial nail 2 is attached to the fingernail by an instantaneous adhesive agent 4 with the thermoplastic resin coating material 3 in between. However, the present invention is applicable to a manicure. In other words, a manicure can be applied over the film of a thermoplastic resin coating material 3 that has been coated on the fingernail 1. The manicure can be removed by way of merely immersing the fingers, on which the manicure has been applied on the coated thermoplastic resin coating material, in hot water at 42° C. to 43° C. for approximately five (5) minutes. Since the film of the thermoplastic resin coating material 3 softens and dissolves, the manicure can be removed without scratching the fingernail; and since no remover or removing solution is required for removing the manicure, roughness of the fingers can be avoided.

In the present invention, as described above, a fingernail is coated with an aqueous type thermoplastic resin coating material, thus allowing this thermoplastic resin coating material to make a dried film. Afterward, an artificial nail is bonded to the fingernail by means of an instantaneous adhesive agent from above this film. Since the film is superior in terms of adhesion to the fingernail, and since the artificial nail is bonded to the fingernail from above the film using an instantaneous adhesive agent that has a strong bonding strength, the artificial nail can be firmly fastened to the fingernail, and an attachment strength which is sufficient to prevent stripping of the artificial nail by ordinary daily activities or light work in water is obtained.

Furthermore, when the fingertip is immersed in hot water for a certain period of time, the film of the thermoplastic resin coating material softens and dissolves; and as a result, the artificial nail can easily be removed without scratching the fingernail. Since there is no damage to the artificial nail, the artificial nail can be reused repeatedly. Furthermore, since no remover or removing solution is used, no resulting roughness of the fingers would occur.

1. An artificial nail attachment method comprising the steps of:
coating a fingernail with a thermoplastic resin coating material, and
after said thermoplastic resin coating material has dried, bonding an artificial nail to said nail by means of an instantaneous adhesive agent from above said thermoplastic resin coating material.

2. The artificial nail attachment method according to claim 1, wherein said thermoplastic resin coating material is of an aqueous type.

3. A solution used for attaching an artificial nail to a fingernail, wherein said solution is a thermoplastic resin coating material comprising 1 to 5 wt % of polyvinyl alcohol added to an ethylene-vinyl acetate copolymer emulsion.
